(19) United States
(12) Patent Application Publication
(10) Pub. No.: US 2014/0274978 A1
(43) Pub. Date: Sep. 18, 2014

(54) Phytosterol spirostane and spirostene derivatives having a wide variety of utilities in humans and other animals

(71) Applicant: Andre Armel Fasciola, Tucson, AZ (US)

(72) Inventor: Andre Armel Fasciola, Tucson, AZ (US)

(21) Appl. No.: 13/999,672

(22) Filed: Mar. 17, 2014

Related U.S. Application Data
(60) Provisional application No. 61/852,143, filed on Mar. 15, 2013.

(51) Int. Cl. A61K 31/58; A61K 31/4045; A61K 45/06

(52) U.S. Cl. CPC A61K 31/58; A61K 45/06; A61K 31/4045
USPC 514/170

(57) ABSTRACT

The present invention utilizes phytosterol, phytoecdysteroid, sapogenin, triterpene, terpene, spirostane, spirostene and androstene derivative compounds to provide a wide variety of utilities to humans and other animals. The compounds and methods provide numerous and wide-ranging benefits including biochemical and constructive metabolic benefits including increases in protein synthesis and synthesis efficiency, ergogenic, adaptogenic, cosmetic and medicinal skin agents and veterinary utilities. The compounds provide agents that increase physical endurance and work output, act as a general tonic for increasing health and vigor, increase sleep time and sleep efficiency and act as rejuvenating agents to reverse the effects of fatigue and stress. Novel utilities for pet, farm, zoo and companion animals are also provided.

### CROSS-REFERENCE TO RELATED APPLICATIONS

This application claims the benefit of U.S. Provisional Application No. 61/852,143, filed Mar. 15, 2013, herein incorporated by reference in its entirety.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

The present invention relates to novel utilities and methods of utilizing phytosterols and the structural and functional analogs and derivatives of phytosterol, phytoecdysteroid, sapogenin, triterpene and spirostanic compounds. More particularly, the invention relates and pertains to non-hormonal spirostane and spirostene anabolic, adaptogenic, ergogenic and cosmetic compositions, methods and utilities.

2. Description of the Related Art Including Information Disclosed Under 37 CFR 1.97 and 37 CFR 1.98

Ecdysteroids are polyhydroxylated steroid compounds of the cholestane series with specific structural features. Ecdysteroids have moulting/metamorphosis activities in arthropods including insects. Phytoecdysteroids, which are plant ecdysteroid structural analogues of the insect moulting hormone ecdysterone (β-ecdysone), have been found to occur in more than 100 plant families. Ecdysteroids have also been identified in fungi, algae, non-arthropod invertebrates and marine organisms.

Plants have survived assaults from organisms and environmental stress for millennia. They have done so by various mechanisms including physical barriers such as thorns and thick cuticles, and by synthesizing an array of defense chemicals as well as other growth and defense mechanisms. Protective mechanisms used by animals and humans are often similar in strategy and complementary in the substances used for protection; substances which activate or modulate protective, metabolic or other pathways in plants may active similar or distinct pathways in animals to their benefit.

Various ecdysteroids have also been shown to possess certain common desirable biochemical and non-hormonal properties in mammals, including humans. Phytosterols including ecdysteroids have been found to have many pharmacological effects in animals and humans such as increasing acetylcholine esterase activity in the brain, reducing the hypoglycemic response to exogenous glucagon and decreasing hepatic cholesterol by stimulating excretion of cholesterol in the bile. These effects have important meaning for pathologies related to dysfunctional stress adaptation which has been hypothesized to be caused by excess or deficient release of stress mediators such as cortisol. It is possible that the metabolism of phytosterols in animals and humans may produce metabolites that are structurally similar to endogenous steroids. The contribution of phytoecdysteroids is likely an important factor in the mechanism of action of many medicinal plants.

While ecdysteroids were first discovered in insects, certain plant species were subsequently found to be far better sources of ecdysteroids—some plant parts contain up to 1.5% ecdysterone by dry weight. Even so, extraction procedures are highly inefficient due to the hydrophilic nature of the ecdysteroid molecule (extraction of 140 kg of dry Lueza seeds containing 1.4% ecdysterone yielded only 6 grams of 96% pure ecdysterone) and, as a result, purified ecdysterone (suitable for cosmetic use) is extremely expensive to produce. Synthetic ecdysterone is similarly expensive to produce. Conversely, analogs of ecdysteroids can be commercially synthesized from inexpensive sapogenin compounds such as diosgenin or agigenin in high purity, albeit in moderate yields. This makes them attractive commercial alternatives to the ecdysteroids for certain types of utilities since they can possess similar, or even superior, biochemical properties and they can be synthesized in high purity on a commercial scale at a reasonable cost.

Historically, there have been innumerable attempts to create “pure” anabolics by structurally modifying the testosterone molecule in order to increase its anabolic activity while reducing its undesirable androgenic (masculinizing) properties.

[chemical structure]

### Testosterone

See, e.g., U.S. Pat. No. 2,521,586 (androstenediol); U.S. Pat. No. 2,843,608 (bolandiol); U.S. Pat. Nos. 2,953,582 and 2,933,510 (clostebol); U.S. Pat. No. 2,900,398 (methandrostenolone); U.S. Pat. No. 2,698,855 (nandrolone); U.S. Pat. No. 3,060,201 (oxymesterone); Ger. Pat. 1,023,764 (Methenolone); Ger. Pat. 1,096,356 (stenbolone); and Brit. Pat. 1,035,683 (trenbolone). These patents disclose compounds of the androstane and androstene series which, by virtue of various structural modifications, possess increased anabolic activity and reduced androgenic activity over the parent compound testosterone.

Mammals commonly inactivate steroidal anabolics by oxidation at C-17. In response to this known metabolic pathway, those skilled in the art typically added an alpha C-17 methyl group to conventional steroid anabolics to sterically hinder this oxidation and increase the steroid's activity.

Side effects of synthetic androgenic steroids include acne, increased facial and body hair, impotence, prostate enlargement, gynecomastia, high blood pressure, liver failure, and neurological problems such as aggressive moods, delusions and paranoia. In contrast, phytosterols (ecdysteroids) have not shown any side effects in humans or animals. Slama and Lafont (1995) summarized toxicity studies, concluding that phytosterols have no toxic response in animals.

Typical anabolic steroids (i.e., testosterone derivatives) accelerate DNA dependent RNA synthesis while ecdysteroids and their functional analogs stimulate protein synthesis by an unknown and independent mechanism.

Compounds structurally similar to those of the invention have been disclosed (see Czech. Pats. 101,826 and 101,827); however, these compounds possess a C-17 beta hydroxyl protected by an alpha C-17 methyl group. This prevents them from being oxidized at C-17 to metabolites capable of ecdysteroid-like activity.

The half-life of the spirostane compounds appears to be much longer than that of the ecdysteroids. Labeled diosgenin (25 mg/kg) in rats had a serum half-life of approximately 14 hours. See Atherosclerosis, 1979, v.33, pp. 71-87. In contrast, labeled ecdysterone (50 mg/kg) in mice had a serum half-life of only 8.15 minutes. See CA 108:16490u. Because diosgenin compounds have a longer half-life in mammals, they are probably more bioavailable since they persist in the body for a much longer period of time. Interestingly, diosgenin administered to humans (3 grams/day for 30 days) was not metabolized in the B-ring. See Atherosclerosis, supra, p. 84. Consequently, the addition of a ketone to the B-ring (such as 7-keto-diosgenin, a compound of the current invention) should have no effect on the human body's metabolization of the compound.

Russian research has disclosed that certain modified natural spirostanes display anabolic activity similar to ecdysterone in male and female rats. These studies did not demonstrate a common mechanism for the anabolic activity of the spirostanes and ecdysteroids, nor was any further research done to determine what similar or other effects the spirostanes might display in higher mammals, including man.

The mechanism by which ecdysteroids exert their anabolic effects has not been elucidated. In fact Russian scientists recently reported that protein stimulation by ecdysterone and turkesterone was completely insensitive to actinomycin D, which represses DNA dependent RNA protein synthesis induced by conventional androgenic/anabolic steroids. See Biol. Nauki (Moscow), 1984, v.11, pp. 16-20. However, Japanese researchers reported that actinomycin D partially repressed protein synthesis induced by ecdysterone. See Chem. Pharm. Bull., 1969, vol. 17, No. 1, pp. 75-81. The inventor hypothesizes it is likely that the more recent Russian research is correct with respect to whether actinomycin D can partially suppress ecdysteroid induced protein synthesis.

The only known ecdysteroid which is a member of the androstane series is rubrosterone (5β-androst-7-en-2β,3β,14α-triol-6,17-dione); rubrosterone has been shown to possess anabolic activity similar to other known ecdysteroids. See Cham. Pharm. Bull., 1968. v.16, pp. 2426-29.

[chemical structure]

### Rubrosterone

In contrast to typical androgenic anabolic steroids which all require a beta hydroxyl at C-17 for anabolic activity, rubrosterone possesses a ketone at C-17. Thus, it would appear that for an androstane series compound to function in the ecdysteroid protein synthetic system, a ketone at C-17 (in addition to a B-ring ketone) is structurally required.

The 3β-hydroxy-6-oxo moiety has been found in several naturally occurring sterols including brassinosteroids (growth phytohormones, i.e. teasterone, one of the most widely distributed brassinosteroids in the plant kingdom, cathasterone and 6-oxocampestanol) and laxogenin. Laxogenin is a naturally occurring compound which was first isolated from Smilax sieboldi (a climbing shrub of the lily family) and later isolated from numerous plants of various plant families. Laxogenin became of interest due to its growth promoting activity in plants and as an analogue of brassinosteroids.

Some of the most common oxysterols are those with a ketone function at carbon-7. Most of these are the 7-keto-Δ5-sterols, which are derived from the oxidation of Δ5 sterols. These compounds can be found in animal tissues, food products and various folk medicines and many are significant inhibitors of HMG-CoA reductase, sterol synthesis and cell replication.

Adaptogens are broadly defined as metabolic regulators that improve the body's ability to adapt to environmental factors and stress, including physiological and psychological stress and aging. Adaptogens must be innocuous and nontoxic, at least in reasonable does and have a nonspecific action or response (which may be in addition to specific actions or responses). Adaptogens must have a normalizing action, restoring the natural homeostasis or state of balance.

### BRIEF SUMMARY OF THE INVENTION

This invention is directed toward phytosterol, phytoecdysteroid, sapogenin, triterpene, terpene, saponin and spirostanic compounds useful as nutraceutical (dietary supplement) non-hormonal anabolic agents, “addaptogenic” agents, ergogenic agents that increase physical endurance and work output, rejuvenating agents that help reverse the effects of aging, agents which improve total sleep time and efficiency and bioactive skin agents in animals including humans.

Adaptogens are reputed to have an anti-stress effect mainly towards stresses of a non-infectious agent. In this aspect adaptogens differ from the spirostanic spirostane and spirostene “addaptogens” (from “additive” and “adaptogens”) or “adaptogenins” (after “adaptogen” and “laxogenin”) of the current invention, which have broad-spectrum immuno-enhancing and immuno-stimulating properties that are also useful to restore homeostasis, normalize functions, enhance protein synthesis and improve numerous other biochemical and physiological functions by -up-regulation and down-regulation of genes and biochemical systems as needed. The general purpose of adaptogens is the reduction of stress reactions in the alarm phase, thereby avoiding the exhaustion stage and providing a certain protection against stress and rebuilding the body after stress or fatigue. Addaptogens or adaptogenins of the current invention also provide protection prior to and during the alarm phase and exhaustion stage as well as providing numerous benefits after stress or fatigue. Without being bound to any theory, the inventor hypothesizes the addaptogen/adaptogenin compounds of the present invention stimulate and increase protein synthesis by initiating transcription and/or increasing the synthesis of messenger RNA (“mRNA”), enhancing the translation of mRNA and further increasing the efficiency of the protein synthetic process by stimulating the production of translational RNA, augmenting the recovery of mRNA synthesis and/or elevating acetyl cholinesterase activity. Similar advantages may be present with regard to microRNAs (miRNA).

Phytosterols, their structural and functional analogs and derivatives and their metabolites may be utilized in practicing the invention. Of particular use in practicing the invention are compounds of the following formulas:

[chemical structure]

(I) where R is H or COCH₃; Q₁ is methylene (—CH₂—), Q₂ is α-H, β-H, or α-OH; Q₃ is carbonyl; Q₄ is methylene and Q₅ is C or Q₄-Q₅ is ethylene(C═C); and C₂₅ is R or S;
(II) Where R is H or COCH₃; Q₁-C₅ is ethylene; Q₃ is carbonyl; Q₄ and Q₅ are methylene or Q₄-Q₅ is ethylene, and; C₂₅ is R or S; and
(III) Where R is H or COCH₃; Q₁ is methylene; C₅-Q₃ is ethylene; Q₄ is carbonyl; Q₅ is C, and C₂₅ is R or S.

Based on clinical testing, particularly preferred are the following spirostanic sapogenins, wherein the carbons are numbered as is usual for spirostanic structures as below:

[chemical structure]

### Spirostane Carbon Numbering

Laxogenin=(25R)-3β-hydroxy-5α-spirostan-6-one;

[chemical structure]

### Laxogenin

Laxogenin acetate=(25R)-3β-acetoxy-5α-spirostan-6-one;
5α-hydroxy-laxogenin=(25R)-spirostan-3β-5α-diol-6-one;

[chemical structure]

### 5-HYDROXY-LAXOGENIN

5α-hydroxy-laxogenin acetate=acetates (25R)-spirostan-3β-acetoxy-5α-ol-6-one and (25R)-spirostan-3β-ol-5α-acetoxy-6-one and/or diacetate (25R)-spirostan-3β-5α-diacetoxy-6-one
7-keto-diosgenin=5,25R-spirosten-3β-ol-7-one;

[chemical structure]

### 7-KETO-DIOSGENIN

7-keto-diosgenin acetate=5,25R-spirosten-3β-acetoxy-7-one

Also preferred are the following spirostanic compounds and their acetates:
(25R)-spirostan-7-en-3β-5α-diacetoxy-6-one;
(25R)-5α-spirostan-2α-3β-5α-triol-6-one;
(25R)-spirostan-3β-5α-15-α-triol-6-one;
(25R)-spirostan-2α-3β-15-α-triol-6-one;
(25R)-spirostan-3β-5-15-α-triol-6-one;
(25R)-5α-spirostan-3,6-dione;
(25R)-3β-hydroxy-5α-spirostan-6-one;
(25R)-hydroxy-5α-spirostan-3β,6-diol;
Laxogenin=(25R)-3β-hydroxy-5α-spirostan-6-one;
(25R)-5α-hydroxy-spirostan-3,6-dione;
(25R)-spirostan-3β-7-diol-6-one;
7-keto-diosgenin=5,25-R-spirosten-3β-ol-7-one;
5,25R-spirosten-3,7-dione
20-Hydroxyecdysone, also known as Ecdysterone or 20E;
7-keto-DHEA;
DHEA=3β-hydroxyandrost-5-en-17-one or 5-androsten-3β-ol-17-one;
6-keto-diosgenin=25R-spirost-4-ene-3,6-diol;
25R-spirost-4-ene-3,6-dione;
   and their derivatives including isomers, diasteromers, configurational isomers and optical isomers thereof, both alpha and beta and R and S, and also including conformational isomers, cis-trans isomers, geometric isomers and stereoisomers and physiologically active salts, esters, ethers and glycosides that are known to those of skill in the art, as well as compounds with protecting groups linked to hydroxy radical or hydroxyl substituent and convertible thereto by hydrolysis, including also pro-compounds (metabolic precursor compounds) and metabolites of the compounds of the current invention.

Examples of such hydrolyzable groups or protecting groups include hydroxyl groups esterified with an acid selected from the group consisting of (i) normal or branched, saturated or unsaturated aliphatic acids containing from 2 to 24 carbon atoms, particularly acetic acid, (ii) aromatic acids containing from 7 to 15 carbon atoms, (iii) dicarboxylic acids containing 3 or more carbon atoms in which only one of the carboxyl groups is esterified to the hydroxyl group(s) on the sterol, and (iv) inorganic acids such as sulfuric and phosphoric acids, the ethers with organic alcohols and inter-molecular ethers, and the glycosides, particularly O-glycosides, of the spirostanes and spirostenes, including mono-glycosides, di-glycosides and tri-glycosides, including both di-glycosides and tri-glycosides where the sugar moiety is linked to two different hydroxyls and the glycosides where two sugars are linked subsequently.

The compounds of the present invention also include derivations, derivatives and analogs of a moiety known to those of skill in the art that may serve a variety of beneficial functions including stabilization of the sterol, solubility and affecting the rate of absorption of the sterol.

The compounds of the present invention can be synthesized from, for example, diosgenin, DHEA and numerous other precursors by methods known to those of skill in the art.

The present inventor has found that 7-keto-diosgenin is unexpectedly synergistic in effects with laxogenin and/or 5-hydroxy laxogenin (which do not appear to synergize with each other). A 1:1 ration of 7-keto-diosgenin to laxogenin and/or 5-hydroxy-laxogenin has been found to be effective; ratios from 1:10 to 10:1 may exhibit greater or lesser synergism.

In addition to the intrinsic anabolic and other activities of the spirostane and spirostene derivatives of the current invention, they also synergistically potentiate the anabolic actions of androgenic anabolic hormones while mitigating their side effects. Laxogenin, 5-hydroxy-laxogenin, 7-keto-diosgenin and their derivatives such as acetates are all particularly useful and preferred in this regard.

Furthermore, the present inventor has found that 7-keto-diosgenin, 5-hydroxy-laxogenin and laxogenin are all synergistic with anabolic substances, including anabolic steroids, particularly the synergistic combination of 7-keto-diosgenin with 5-hydroxy-laxogenin or laxogenin, with anabolic substances including anabolic steroids. The inventor has further found, that in general, spirostanes and spirostenes are synergistic with each other, and that the spirostanic phytosterol compounds and their synergistic spirostane/spirostene combination are synergistic with anabolic steroids, while mitigating and ameliorating the side effects of those steroids (which have many pharmaceutical uses).

The invention comprises methods for utilizing known and novel structural and functional analogs and derivatives of phytosterols (such as diosgenin); these compounds have novel utilities in humans and animals as non-hormonal anabolics, non-hormonal cosmetic agents, as agents that increase physical endurance and work output, as agents which act as a general tonic for increasing health and vigor, as agents which improve total sleep time and efficiency and as rejuvenating agents to help reverse the effects of aging. Effects include pro-anabolic, antioxidant, hepatoprotective and hypoglycemic effects.

The compounds of the current invention demonstrate these novel utilities in vertebrate animals including mammals, carnivores, omnivores, herbivores, pets including cats and dogs and companion animals, farm and produce animals, laboratory animals, zoo animals, reptiles, fish, birds and humans. The compounds of the present invention may also be useful for bees, including those suffering from pesticides, viruses or at risk from Colony Collapse Disorder, but it is possible the ecdysone analogs in particular may interfere with mating and molting due to some similarity to insect and arthropod hormones.

Additionally, the compounds of the invention in therapeutically effective amounts have novel utility as skin agents for all animals and as cosmetic agents for humans which accelerate the formation of a broad range of proteins within the skin while expressing minimal hormonal activity.

Advantages, objects and results of the present invention include:
1) An increase in body protein synthesis;
2) An increase in efficiency (not amount of hormone) of the translation of mRNA—thus increasing protein synthesis efficiency; prevents degradation of protein synthesis efficiency, augmentation of the recovery of mRNA synthesis, elevated acetylcholinesterase activity;
3) Ability to restore, repair, rejuvenate, regulate, function as a protective aid, build muscle, improve overall function, restore homeostasis and improve wound healing;
4) Anti-inflammatory reduction or pro-inflammatory increase as needed, restores suppression of IL-1beta induced activation of NF-kB and PI3K-Akt pathway, restores homeostasis and normal functioning of skin via both pro-inflammatory and/or anti-inflammatory effects as needed, skin metabolism-activating, blood flow improver, anti-wrinkle, inhibition of psoriasis and eczema;
5) Antioxidant, antiradical, increases antioxidant activity;
6) Hypoglycemic (exogenous glucagon);
7) Neuroprotective, neuroregenerative, reduction of beta-amyloid (βA) deposits, protection of cerebral neurons, inhibition of production of lipid peroxides in brain tissue;
8) Increased lipid, carbohydrate and protein metabolism;
9) Anti-diabetic—reduction of blood sugar levels/increases insulin sensitivity, enhancement of carbohydrate metabolism;
10) Enhancement of immune system, restores homeostasis via both immune-stimulating and immune-suppressant actions to normalize functions;
11) Anti-Histamine, inhibits induced histamine release;
12) Increase stores of glycogen and pyruvate acid, decreases lactic acid;
13) Decreases urea in blood, restores renal function, decreases renal dysfunction;
14) Increases blood flow by reducing pressure in arterial walls, antiarrythmic;
15) Hypocholesterolemic, reduces cholesterol levels;
16) Stabilizes and normalizes proteins and phospholipids;
17) Increase in caloric carbohydrate reserve in muscle;
18) Increase in contractility of cardiac muscle and stroke volume;
19) Anabolic (as opposed to catabolic), “building up” of tissues and organs, anabolic utilities including increased muscle mass, prevention of loss of muscle mass, mitigates damage of catabolism, works via mechanism other than anabolic receptor(s);
20) Spirostanes and spirostenes are synergistic with each other, both are synergistic with anabolic steroids, androstanes and androstenes;
21) Replacement of all anabolic steroids, safe alternative to anabolics while providing all utilities of anabolics, remediation, repair, treatment, mitigation, amelioration and cure of long term anabolic use and damage;
22) Cytotoxic to tumor cells, cataracts;
23) Stimulation of proton gated ions, Ca2+, Mg2+;
24) Organ regeneration from trauma and chemical damage;
25) Removal of fatigue, improve physical condition, CNS stimulant, useful against fatigue by people who suffer asthenic states and by healthy people who show astheny during periods of high mental exertion or after intensive physical exertion;
26) Combats states of stress, both biological and environmental, decreases the expression of stress-reaction;
27) Increase acetylcholinesterase;
28) Interaction with numerous cell receptors;
29) Altering gene expression in plants to protect from insects, accelerating rate of plant growth, grow better, insect resistant crops, turns on 1000's of known unknown PES in plants;
30) Adaptogenic and addaptogenic, treats and restores to normal non-disease states of functioning, rejuvenates, reinvigorates, restores health, homeostasis and a normal state of functioning, immunomodulatory;
31) General tonic, improves physical endurance and work output, increases health and vigor, rejuvenates, restores, reverses effects of aging; amelioration of psychological and physiological depression, enhancement of libido and sexual function, enhancement of mental and physical function, possible amelioration or mitigation of dementia-related diseases;
32) Improves stress markers via addaptogenic processes;
33) Improves the growth rate for nails and hair;
34) Improved productivity in farm animals including weight gain and improved wool growth; and
35) Combats “failure to thrive” in companion animals, particularly elderly animals with disease or chronic conditions, improves quality of life for companion, working and farm animals.

The spirostanic derivative compounds of the present invention have an “addaptogenic” or “adaptogeninic” effect that serves to restore homeostasis and normal functioning as opposed to being “pro” or “anti.” Thus they stimulate adaptation to any kind of stress via gene regulation and stimulate anabolic (constructive metabolic) functions, such as protein synthesis, only when and where such stimulation is needed. Similarly, they may provide immuno-stimulating or immuno-suppressant effects, anti-inflammatory or pro-inflammatory effects, etc. The compounds of the present invention have ergogenic effects (increasing capacity for bodily or mental labor, particularly by eliminating fatigue symptoms) that similarly partake of a normalization and maximization of the various anabolic and catabolic metabolic pathways.

With regard to skin preparations, the adaptogenins of the present invention may be pro-inflammatory or anti-inflammatory as needed to restore normal functioning and homeostasis and that may strengthen or diminish the immune response as needed. For example, eczema and psoriasis are examples of hyper inflammatory skin disorders for which targeted anti-inflammatory compositions may be indicated. Conversely, in the elderly with skin that has thinned and may be more easily damaged and has a reduced or diminished blood flow and resultant reduced or diminished immuno-response, topical treatments that inhibit skin inflammation may be contraindicated. The sapogenin derivative compounds of the current invention are unique in exhibiting both pro-inflammatory and anti-inflammatory effects and are thus useful for both the hyper inflammatory disorders and the disorders of age including diminished immune response. The compounds exhibit analogous immunomodulatory effects, providing immuno-stimulating or immunoenhancing effects and immuno-suppressant effects as needed to restore homeostasis.

Addaptogens are thought to interact with stress response mediators and decrease cellular sensitivity to stress in various ways including interaction with DNA and RNA, hormones and other neurotransmitters, receptors, cytokines and other biochemical mechanisms. Adaptogens and the adaptogenins likely increase and/or decrease production of specific G-protein coupled receptors (GPCRs) via the DNA-RNA transcriptional system, with GCPR genes and mediators signaling pathways and up regulating and down regulating genes as needed to respond to stress and restore homeostasis. Stress response mediators, including molecular chaperons, G protein-coupled receptors and their mediators, stress kinase JNK, FOXO3, DAF-16, HSF1, neuropeptide Y, cortisol, estrogens and nitric oxide, play important roles in ageing and senescence.

The present invention further provides these and other advantages. It is to be expected that the number of sub-inventions and applications obvious to those skilled in the relevant arts is limited only by imagination and time, and any such derivative inventions and applications should be considered to be part of the invention disclosed herein. Still further objects and advantages of the present invention will become more apparent from the following detailed description and appended claims.

Before explaining the disclosed embodiments of the present invention in detail, it is to be understood that the invention is not limited in its application to the details of the particular products and methods illustrated, since the invention is capable of other embodiments, including those embodiments that have not yet been reduced to practice and tested. In addition, the terminology used herein is for the purpose of description and not of limitation.

### DETAILED DESCRIPTION OF THE INVENTION

This invention pertains to phytosterol, phytoecdysteroid, sapogenin, triterpene, spirostane, spirostene, compounds which possess novel utilities as constructive anabolics, veterinary and cosmetic agents, adaptogens, ergogenics and provide enhancement of numerous beneficial biochemical pathways and mechanisms, resulting in multiple “quality of life” improvements.

It is an object of this invention to provide spirostane and spirostene (“spirostanic”) phytosterol compounds which initially act as conventional anabolic agents by stimulating DNA dependent RNA synthesis and protein synthesis, and which can then be oxidized by normal enzymatic pathways to a C-17 metabolite conferring ecdysteroid-like activity.

Spirostane and/or spirostene compounds of the present invention are useful as non-hormonal anabolic agents which increase athletic performance and work output in humans and other mammals in doses of 1-60 mg three times per day. In young human volunteers (ages 18-30) receiving 10-20 mg/day of the phytosterol derivative compounds of the present invention in combination with physical training, subjects reported an increase in appetite, accelerated strength and weight gain, and a virtual elimination of lactic acid formation during intense exercise (such as weight resistance training). Work output before failure was also reported to be increased substantially.

The spirostanic compounds of the invention are also useful in increasing and restoring health and vigor, useful in preventing fatigue, and useful in improving physical condition and performance and increasing work output in aging humans and other mammals. For example, 15 mg of 25R-Spirostan-3B, 5α-diol-6-one was given to a 65 year old woman in three divided doses per day (t.i.d.) for two weeks. She reported an overall increase in well-being, accompanied by a definite increase in energy and physical stamina which permitted her to work 2-3 times as long in her garden. One 80 year old male and one 79 year old female volunteer, both receiving 60 mg/day of the same compound, reported definite increases in physical energy and stamina within one week of beginning administration. The couple also found that their total sleep time increased from 6 hrs/night to approximately 8 hrs/night over the course of one month and that this increased sleep was more restful.

Additionally, the phytosterol compounds of the present invention are useful as anabolic adjuncts to conventional anabolic steroids. Typical androgenic anabolic steroids increase protein synthesis by accelerating DNA dependent RNA synthesis. Compounds of the invention appear to act analogously to ecdysteroids in that their anabolic effects stem from an unknown mechanism which makes the overall protein synthetic process more efficient—while actinomycin D represses DNA dependent RNA protein synthesis induced by conventional androgenic/anabolic steroids, actinomycin D was unable to suppress ecdysteroid induced protein synthesis, indicating that the ecdysteroids act by some unknown and independent mechanism.

None of the sapogenin compounds tested in humans has displayed any hormonal activity. Competitive binding inhibition studies conducted at Baylor University in Texas showed that 25R-Spirostan-3β, 5α-diol-6-one did not bind to androgen or estrogen receptors even at the limits of compound solubility. Consequently, the spirostane and spirostene compounds of the present invention are expected to be devoid of hormonal activity.

Preliminary testing using a combination of 6-keto sapogenin derivative laxogenin (30 mg/day) and 5-androsten-3β,7β,17β-triol (30-50 mg/day) (a natural metabolite in human skin) shows a synergistic effect.

Competitive bodybuilders who were currently using various anabolic steroids volunteered to also use 25R-Spirostan-3B, 5α-diol-6-one or 7-keto-diosgenin in divided doses totaling 20 mg/day. These volunteers reported greatly accelerated increases in muscle mass (between 10-20 lbs over two months); these increases were far beyond what they had previously experienced using only conventional anabolic steroids. Therefore, the anabolic effects of the sapogenin compounds of invention are unique and show surprising and unexpected results in that they are synergistically additive or multiplicative to the protein synthetic effects and muscle-building effects of conventional anabolic steroids.

The amount of phytosterol derivative required for the described utilities depends primarily on the age and weight of the subject. Younger subjects require lower daily doses; older subjects require somewhat greater doses. In general, for laxogenin, 5-hydroxy-laxogenin and 7-keto-diosgenin, one can take the age of the subject, divide by two, and resulting number is an effective oral daily dose in milligrams for a person of average body weight (70 kg/154 lbs). The dose can be proportionately increased for persons of greater than average body weight. Effective doses in humans preferably range from 1-180 mg/day for the phytosterols of invention, more preferably 6-60 mg/day, and should preferably be taken in 2-3 equal doses per day. Administration of these compounds can be in any form that permits systemic absorption, but preferably in an orally administered controlled dose form such as a tablet, capsule, powder, sustained release formulation, sublingual, suspension, solution, spray, syrup, dispersion or other forms known to those of skill in the art. For skin preparations, much lower doses can be utilized as the compounds of the present invention are readily and directly absorbed. Doses in skin preparations are preferably 0.01-5 mg./day and more preferably 0.1-1 mg./day.

The compounds of the present invention also have the ability to mediate and/or reduce pain in humans and other animals. A man in his 60's received a double knee replacement due to long time injuries. He began taking 3 doses per day of Laxogenin at 150 mg per dose one week prior to knee surgery. He stated his pain had diminished his third day on Laxogenin-2 days before his operation. The day after his operation the patient was able to discontinue his morphine drip altogether. He was able to began light walking 3 days after surgery, by the 7th day he could stand and walk far distances free of pain, stiffness and discomfort. He applied a liquid version of Laxogenin topically and reduced surface inflammation and scarring greatly.

The spirostanic compounds of the present invention are also particularly useful in mammals that are dedicated carnivores, omnivores or herbivores, especially dogs, cats and other companion animals, working animals, farm animals and zoo animals, to increase health and vigor and help reverse many of the effects of aging. For example, when administered to feedlot cows, it is expected the compounds of the current invention will increase lean muscle mass; the volume of protein in feed may be reduced by 10% while still achieving the wait gain of the normal 100% of protein feed, or additional weight gain is to be expected from the normal feed. For racehorses and competitive horses, in addition to the physical and health optimizing benefits, the current compounds mitigate any issues of anabolic treatments or anabolic damage. For egg-laying chickens, it is expected that, for example, the number and/or size of eggs, the size of yolks, nutritional content and other desirable properties will increase. Similar results are expected for fish and fish eggs, including farm and aquarium fish. It is also expected that fish and other animals will grow faster. The compounds of the current invention are preferably administered in two divided doses preferably ranging from 1-200 mg/day, more preferably 1-25 mg/day, most preferably 1-10 mg/day.

Two older Rhodesian Ridgeback dogs at age 13 were overweight, weak and ailing (exhibiting “failure to thrive”); they would struggle to get up from their beds and could no longer take long walks. After administration of either Laxogenin or 7-keto-diosgenin or a combination of the two, the dogs started to exhibit puppy like behavior after 48 hours. Their appetite and activity increased, they became leaner—leaner muscle, wanted to go for walks and became like a new dog. They lived another 3 years on a daily dosages to age 16. Their treating Veterinarian said he had never seen dogs of their size and breed live to such an advanced age. Numerous similar reports were collected.

In dogs of 7-14 years of age, 25R-Spirostan-3β, 5α-diol-6-one or 7-keto-diosgenin administered in two divided doses ranging from 1-10 mg/day, increased activity, playfulness, physical stamina, appetite and interaction with owners, reduced stiffness and weakness, and improved overall health were reported by pet owners. This rejuvenating effect was surprising because 25R-Spirostan-3B, 5α-diol-6-one has been shown to produce reduced anabolic activity in older (i.e. mature) rats, and higher anabolic activity in young rats. See Kokl. Akad. Nauk. Uzb., SSR, 1981, v.3, pp. 31-33. See also Farmakol. Toksikol., 1976, 39(5), pp. 631-35. In addition, over 2-3 months administration, the compounds increased many of the dogs' lean muscle mass while reducing body fat

The compounds of invention are also useful for lengthening the life span of various large breeds of dogs (and presumably small breeds). One Great Dane. given 25R-Spirostan-3B, 5α-diol-6-one daily (8 mg/day) from age 8 on. lived to be almost 13 years old. The dog had to be euthanized because it suffered from dysplasia in both hips; otherwise it was healthy and active. The upper limit of age for Great Danes of this lineage was previously considered to be 10 years.

The compounds of invention are also useful for increasing physical stamina and endurance in young working and sporting dogs used for hunting, sled pulling and herding and dogs in competitions such as greyhound racing, field trials, agility, disc dog, dock jumping and Schutzhund. Owners of hunting dogs reported noticeable improvement in their dogs physical endurance within one week of administration of 5-10 mg/day of 25R-Spirostan-3B, 5α-diol-6-one or 7-keto-diosgenin.

The sapogenin compounds are preferably administered 2-3 times per day, in total daily doses preferably ranging from 5-60 mg/kg. These compounds can be administered in any form that permits systemic absorption, but preferably in an orally administered controlled dose form as above.

Phytosterol derivative compounds of the present invention are also useful as skin agents for animals and useful as cosmetic agents when formulated in preparations which, when applied to the skin, create a penetrating flux that will deliver the compound to both the epidermis and dermis. The preferred lotion formulations are of low irritancy and are generally comprised of either a spirostane or a spirostene or, preferably for synergistic reasons, a combination of both [0.05-0.2% (w/w) combined in a ratio of 2:1 to 1:2] dissolved in a solution consisting of propylene glycol [50-70% (w/w)]. propylene carbonate [15-25% (w/w)], water [10-20% (w/w)], and glycerol [0.12.0% (w/w)]. The steroid solutions are most preferably formulated at 80-90% saturation.

The compounds of invention, when formulated as above, are useful for reversing many aspects of degenerative skin disorders (such as thin, fragile, inelastic skin and weakened dermal blood vessels) due to various causes such as aging or chronic corticosteroid therapy. In an elderly person 80 years of age suffering from severely atrophic skin on the dorsal side of both hands (accompanied by traumatic purpurra, and venous lakes), dramatic increases in skin elasticity and strength were seen after only one month of treatment with 25R-Spirostan-3B, 5α-diol-6-one or 7-keto-diosgenin. Virtually all traumatic purpurra and venous lakes were resolved within two months and no new purpurra spontaneously appeared—a clear indication that dermal vascular wall strength had increased substantially by the end of the second month of treatment.

Spirostanic phytosterol derivative compounds of the present invention are also useful as non-hormonal cosmetic agents when formulated in preparations which, when applied to the skin, create a penetrating flux that will deliver the compound to both the epidermis and dermis. The preferred lotion formulations are of low irritancy and are generally comprised of a sapogenin compound of the invention [0.05-0.2% (w/w) basis] dissolved in a solution consisting of propylene glycol [50-70% (w/w)], propylene carbonate [15-25% (w/w)], water [10-20% (w/w)], and glycerol [0.1-2.0% (w/w)]. When formulated for maximum bioavailability (i.e. at 90% steroid saturation), the bioavailability of the steroidal compounds is essentially independent of steroid concentration. In the epidermis, the compounds of invention activate skin metabolism resulting in accelerated keratinocyte and corneocyte formation. This caused the surface of the skin to become visibly smoother and noticeably softer within 2-4 weeks of application in normal human test subjects. in addition, the compounds improved the skin's water vapor barrier as evidenced by test subjects reporting that their skin maintained a “dewy” look.

When compounds of the invention are delivered into the human dermis, they are useful for accelerating the production of proteins including collagen and elastin. In virtually all subjects testing the various lotion formulations, the increase in collagen and elastin formation within the skin was so obvious that when the left and right index fingers were placed tip to tip, the finger that was used to apply the lotion was noticeable thicker (20-50%) than the finger which was not used to apply the lotion. The magnitude of this effect was found to increase with the age of the subject, which correlates well with the fact that the skin loses approximately 10% of its collagen and elastin per decade after the age of 20.

The compounds of invention, when formulated as above in therapeutically effective amounts, are therefore useful for reversing many aspects of degenerative skin disorders due to various causes such as aging, acute or chronic inflammation, disease or chronic corticosteroid therapy. The present compounds and formulations are particularly useful for increasing dermal wall strength for conditions such as thin, fragile, inelastic or atrophic skin, weakened dermal blood vessels, purpurra, venous lakes, irritated and inflamed skin, diaper rash, skin inflammation and visible signs of skin aging, etc. The compounds and formulations are also useful for conditions such as eczema, psoriasis, acne, impetigo, cutis laxa, dermatamyositis and other skin diseases. The compounds and formulas do not produce any irritation, even in cases of allergic contact dermatitis.

As a practical matter it is not always prudent to formulate the compounds at 90% saturation as this causes the formulations to be somewhat temperature sensitive (i.e., if subjected to temperatures below 50° F. the steroid will begin to crystallize out of solution). Consequently, it may sometimes be preferable to formulate the steroid at a lower level of saturation in order to ensure the stability of the solution.

The slight advantage in using a 0.2% concentration of steroid in the formulation (as opposed to 0.1%) is that solution bioavailability is approximately 10-15% greater and is maintained for approximately 10-15% longer; however, this small increase is not statistically significant. See J. Invest. Dermatol., 1982, v.79, no.6, pp. 388-91.

The compounds of invention are also particularly useful as anti-wrinkling agents and for reducing the appearance of existing wrinkles when formulated as above. Because the most noticeable (deep) wrinkles penetrate through the epidermis and partially into the dermis, acceleration of collagen and elastin formation within the dermis has the effect of pushing in on the wrinkle from the sides and up on the wrinkle from below, thus compressing the wrinkle from all angles and making it much less noticeable. Most “deep” wrinkles were reduced in depth by 30-70% within 2-4 weeks of administration in normal human subjects; fine wrinkles were less noticeably reduced (by 20-40%).

When formulated as above, compounds of the invention are useful in promoting nail growth. Subjects applying the formulas to the cuticle area on each finger reported accelerated nail growth, accompanied by an increase in nail strength; subjects that were previously unable to grow long nails were able to grow long nails that did not easily chip or peel.

The compounds of the present invention are also useful in shampoos, conditioners, bathing products, foams, ointments, gels, pastes, dermal patches, etc. They may be formulated in the numerous topical combinations and methods and synergistic formulations known to the art.

In order to achieve the results reported herein, the formulations described above were liberally applied to the skin (as one would a suntan lotion) twice per day, as close to 12 hours apart as possible. Additional applications of up to four times per day (evenly spaced throughout the day) yielded somewhat greater benefits in the few subjects who were able to maintain this regimen; however, this increased frequency of administration is probably not practical or necessary for most persons.

In addition to the human utilities, the compounds and formulations possess similar veterinary skin and hoof or claw utilities for animals, including pets such as cats and dogs, farm animals such as horses, cows and pigs, zoo animals.

The following ingredients may have additive, multiplicative or synergistic effects when combined with the compounds of the present invention, particularly synergistic effects for the methods of providing the advantages of the present invention listed below in conjunction with the synergistic ingredients:
1) L-taurine: Mimics the effect of insulin independent of carbohydrate ingestion, slows muscle tissue breakdown, reduces appetite, increased hypothalamic mTOR activity, maintains or maximizes cellular hydration;
2) Rhaponticum Carthamoids extract (RCE): adaptogenic and ergogenic effects, mitigates the body's ability to convert carbohydrates to fat, increases activity of the muscle cell cytoplasm/ribosome, leading to an accelerated rate of mRNA translation and translocation, elevates intracellular calcium followed by sustained Akt activation and increased protein synthesis, allows for an increase in both the number and activity of ribonucleic acids and the forming of new contractile muscle proteins from available amino acids;
3) Orotic Acid: accelerated skeletal muscle hypertrophy (muscle growth), enhances athletic power, endurance and performance as a function of muscle hypertrophy and energy supply, stimulates the synthesis of DNA and RNA nucleic acids involved in protein synthesis, augments/stimulates protein synthesis, enhances repair and regenerative processes in many tissues, increases ATP production, promotes glycogen storage and increases/optimizes glucose and oxygen metabolism and usage with better glucose disposal, optimizes glycogen storage and glycolysis, resulting in greater VO2 maximum, reduces of cortisol and carbon dioxide, increase of ATP production;
4) Tri-Methyl Glycine (“Betaine”; methyl donor and precursor to Sam-E): control of cell growth, anti-inflammatory properties, improves muscle to fat ratios, improves lipid metabolism, improves liver methylation and repairing, renewing and re-generating of existing lean body mass, improves fat loss mechanisms, improves synthesis new muscle tissue and prevents loss of muscle tissue, improves re-synthesis and repair of RNA, increases anabolic hormones such as GH and IGF-1 and decreases catabolic hormones such as cortisol, improves ability to sustain protein synthesis, decreases AMPK phosphorylation with resultant decreased inhibitory effect on the mTOR pathway;
5) Eurycoma Longifolia, traditionally called Tongkat Ali: promotes health, well-being, strength, stamina and libido, prevention and treatment of anxiety, arthritis, migraine headaches and to improve digestion with low carbohydrate diets;
6) Suma extract: natural adaptogen and anabolic accelerates muscle growth and repair, supports the glandular and immune systems (preventing free-radical damage), relieves pain and fights fatigue;
7) Vitamins, minerals (particularly germanium which increases blood oxygen levels and zinc), amino acids, Allantoin (a regenerative muscle cell builder), pfaffic acids including Beta Ecdysterone, Sitosterol and Stigmasterol;
8) DHEA: blunts excessive Cortisol release increases and/or preserves natural hormone levels;
9) Melatonin: blunts excessive Cortisol release increases and/or preserves natural hormone levels, sleep aid;
10) Leucine: regulates translation initiation of protein synthesis in skeletal muscle after exercise. The decrease in Protein synthesis is associated with inhibition of translation initiation factors 4E and 4G and ribosomal protein S6 under regulatory controls of intercellular insulin signaling and Leucine concentrations as modified by the adaptogenins of the present invention. Leucine's effects on mTOR is synergistic with insulin via the phosphoinositol 3-kinase signaling pathway;
11) Fenugreek, GH, GABA, HGH, Testosterone, diosgenin, ashwagandha root, epimedium, Ginko biloba, Hormone replace therapy, Mucuna Pruriens, Tribulus Terristis, Lueza carthamoides, Seratula coronate, Ajuga turkestanica, Cynotis somaliensis, Rhaponticum uniflorum, Rhaponticum carthamoides, Pfaffia paniculate, Luezea carthamoides, Panex ginseng, turkesterone Fo Ti—Hoshou Wu, Maca Root: useful synergists.

### Example 1

In dogs of 7-14 years of age, 25R-Spirostan-3β, 5α-diol-6-one or 7-keto-diosgenin administered in two divided doses ranging from 1-10 mg/day, increased activity, playfulness, physical stamina, appetite and interaction with owners, reduced stiffness and weakness, and improved overall health as reported by pet owners. In addition, over 2-3 months administration, the compounds increased many of the dogs' lean muscle mass while reducing body fat.

### Example 2

A Great Dane that was given 25R-Spirostan-3B, 5α-diol-6-one daily (8 mg/day) from age 8 on lived to be almost 13 years old. The dog had to be euthanized because it suffered from dysplasia in both hips; otherwise it was healthy and active. The upper limit of age for Great Danes of this particular lineage was previously considered to be 10 years.

### Example 3

Owners of hunting dogs reported noticeable improvement in their dogs physical stamina and endurance within one week of administration of 5-10 mg/day of 25R-Spirostan-3B, 5α-diol-6-one or 7-keto-diosgenin.

### Example 4

15 mg of 25R-Spirostan-3B, 5α-diol-6-one was given to a 65 year old woman in three divided doses per day (t.i.d.) for two weeks. She reported an overall increase in well-being, accompanied by a definite increase in energy and physical stamina which permitted her to work 2-3 times as long in her garden.

### Example 5

One 80 year old male and one 79 year old female volunteer, both receiving 60 mg/day of the same compound, reported definite increases in physical energy and stamina within one week of beginning administration. The couple also found that their total sleep time increased from 6 hrs/night to approximately 8 hrs/night over the course of one month and that this increased sleep was more restful.

### Example 6

Formulate the compounds of the present invention as above and combine with a therapeutically effective amount of melatonin as a sleep aid.

### Example 7

The preferred spirostane lotion formulations are generally comprised of a phytosterol compound of the invention [0.05-0.2% (w/w) basis] dissolved in a solution consisting of propylene glycol [50-70% (w/w)], propylene carbonate [15-25% (w/w)], water [10-20% (w/w)], and glycerol [0.1-2.0% (w/w)].

### Example 8

The preferred spirostane lotion formulations are of low irritancy and are generally comprised of a combination of an androstene and a sapogenin compound of the invention [0.05-0.2% (w/w) combined in a ratio of 2:1 to 1:2] dissolved in a solution consisting of propylene glycol [50-70% (w/w)]. propylene carbonate [15-25% (w/w)], water [10-20% (w/w)], and glycerol [0.12.0% (w/w)]. The steroid solutions are most preferably formulated at 80-90% saturation.

### Example 9

In an elderly person 80 years of age suffering from severely atrophic skin on the dorsal side of both hands (accompanied by traumatic purpurra and venous lakes), dramatic increases in skin elasticity and strength were seen after only one month of treatment with 25R-Spirostan-3B, 5α-diol-6-one or 7-keto-diosgenin formulated as above. Virtually all traumatic purpurra and venous lakes were resolved within two months and no new purpurra spontaneously appeared—a clear indication that dermal vascular wall strength had increased substantially by the end of the second month of treatment.

### Example 10

Several formulas within the range described above were tested by a 62 year old woman with severe allergic contact dermatitis; none produced any skin irritation.

### Example 11

When formulated as above, compounds of the invention are useful in promoting nail growth. Subjects applying the above formulas to the cuticle area on each finger reported accelerated nail growth, accompanied by an increase in nail strength. In particular, one 47 year old female subject (who was previously unable to grow her nails long) reported that, for the first time, she was able to grow long nails that did not easily chip or peel.

### Example 12

Several competitive bodybuilders (25-35 years of age) who were currently using various anabolic steroids volunteered to also use 25R-Spirostan-3B, 5α-diol-6-one or 7-keto-diosgenin in divided doses totaling 20 mg/day. These volunteers reported greatly accelerated increases in muscle mass (between 10-20 lbs over two months); these increases were far beyond what they had previously experienced using only conventional anabolic steroids.

### Example 13

Human volunteers familiar with conventional anabolic steroids used 60 mg/day (20 mg tid) 5-androsten-3β,17β-diol-7-one diacetate orally, in conjunction with weight resistance training. They reported increases in strength and endurance as well as accelerated increases in muscle mass accretion over. conventional steroids. No subject reported any noticeable androgenic side-effects, which are common with testosterone derivatives.

### Example 14

A 44 year old male with total high cholesterol of 270 (LDL 175) reported a significant reduction in total cholesterol levels after a 3 month period using a combination of 7-keto and Laxogenin (5 mg 2× daily). After three months total cholesterol levels were reduced to 230 (LDL 135).

No limitations with respect to the specific embodiments and examples disclosed herein are intended or should be inferred, as the examples and embodiments are representative only. While examples and preferred embodiments of the present invention have been shown and described, it will be apparent to those skilled in the art, or ascertainable using no more than routine experimentation, that many changes and modifications may be made without departing from the invention in its broader aspects. The appended claims are therefore intended to cover all such changes, modifications and equivalents as fall within the true spirit and scope of the invention.

I claim:

1. A composition for increasing anabolic activity, protein synthesis, lipid and carbohydrate metabolism and ion currents in animals comprising an effective amount of a spirostane combined with an effective amount of a spirostene, wherein the spirostane and the spirostene have a synergistic effect.

2. The composition of claim 1 wherein the spirostane is selected from the group consisting of laxogenin, 5-hydroxy-laxogenin, laxogenin acetate, 5-hydroxy-laxogenin acetate and combinations thereof and the spirostene is selected from the group consisting of 7-keto-diosgenin, 7-keto-diosgenin acetate and combinations thereof.

3. The composition of claim 1 wherein the spirostane and the spirostene are selected from the group consisting of compounds of the formula: [chemical structure]
(I) where R is H or COCH₃; Q₁ is methylene, Q₂ is α-H, β-H, or α-OH; Q₃ is carbonyl; Q₄ is methylene and Q₅ is C or Q₄-Q₅ is ethylene; and C₂₅ is R or S;
(II) Where R is H or COCH₃; Q₁-C₅ is ethylene; Q₃ is carbonyl; Q₄ and Q₅ are methylene or Q₄-Q₅ is ethylene, and; C₂₅ is R or S;
(III) Where R is H or COCH₃; Q₁ is methylene; C₅-Q₃ is ethylene; Q₄ is carbonyl; Q₅ is C, and C₂₅ is R or S; and combinations thereof.

4. The composition of claim 1 wherein the spirostane is selected from the group consisting of laxogenin, laxogenin acetate, 5α-hydroxy-laxogenin, 5α-hydroxy-laxogenin acetate and combinations thereof and the spirostene is selected from the group consisting of 7-keto-diosgenin, 7-keto-diosgenin acetate and combinations thereof.

5. The composition of claim 1 wherein the spirostane is selected from the group consisting of (25R)-3β-hydroxy-5α-spirostan-6-one, (25R)-3β-acetoxy-5α-spirostan-6-one, (25R)-spirostan-3β-5α-diol-6-one, (25R)-spirostan-3β-acetoxy-5α-ol-6-one, (25R)-spirostan-3β-ol-5α-acetoxy-6-one, (25R)-spirostan-3β-5α-diacetoxy-6-one and combinations thereof and the spirostene is selected from the group consisting of 5,25R-spirosten-3β-ol-7-one and 5,25R-spirosten-3β-acetoxy-7-one and combinations thereof.

6. The composition of claim 1 wherein the spirostane and spirostene and combined in a ratio from 1:10 to 10:1.

7. The composition of claim 1 wherein the spirostane and spirostene are combined in a 1:1 ratio.

8. The composition of claim 1 wherein the animals are selected from the group consisting of humans, vertebrates, mammals, birds, fish, bees, pets, companion animals, working animals, farm animals, zoo animals and combinations thereof.

9. The composition of claim 1 wherein the effective amount of the spirostane is preferably an amount ranging from 1-200 milligrams per day, more preferably 1-25 milligrams per day and most preferably 1-10 milligrams per day and the effective amount of the spirostene is preferably an amount ranging from 1-200 mg/day, more preferably 1-25 mg/day and most preferably 1-10 mg/day.

10. The composition of claim 1 wherein the effective amount of the spirostane is preferably 5-60 milligrams per kilogram of animal weight per day and the effective amount of the spirostene is preferably 5-60 milligrams per kilogram of animal weight per day.

11. The composition of claim 1 wherein the composition additionally comprises an anabolic agent.

12. The composition of claim 1 wherein the anabolic agent is an anabolic steroid.

13. An anabolic, ergogenic, immunoprotective and rejuvenating addaptogenic composition for animals, the composition comprising an effective amount of a spirostane derivative selected from the group consisting of laxogenin derivatives, 5-hydroxy-laxogenin derivatives and combinations thereof and an effective amount of a spirostene derivative comprising a 7-keto-diosgenin derivative.

14. A composition for improving health and mitigating and ameliorating failure to thrive in animals comprising an effective amount of a spirostane combined with an effective amount of a spirostene, wherein the spirostane and the spirostene have a synergistic effect.

15. The composition of claim 14 wherein the spirostane is selected from the group consisting of laxogenin, 5-hydroxy-laxogenin, laxogenin acetate, 5-hydroxy-laxogenin acetate and combinations thereof and the spirostene is selected from the group consisting of 7-keto-diosgenin, 7-keto-diosgenin acetate and combinations thereof.

16. The composition of claim 14 wherein the animals are selected from the group consisting of humans, vertebrates, mammals, birds, fish, pets, companion animals, working animals, farm animals, zoo animals and combinations thereof.

17. The composition of claim 14 wherein the composition additionally provides mitigation and amelioration of pain and improvement of wound healing

18. A composition for improving quality of sleep comprising an effective amount of a spirostane combined with an effective amount of a spirostene, wherein the spirostane and the spirostene have a synergistic effect.

19. The composition of claim 18 wherein the spirostane is selected from the group consisting of laxogenin derivatives, 5-hydroxy-laxogenin derivatives and combinations thereof and an the spirostene derivative is a 7-keto-diosgenin derivative.

20. The composition of claim 19 wherein the composition additionally comprises melatonin.

21. A composition for improving skin health comprising an effective amount of a spirostane combined with an effective amount of a spirostene, wherein the spirostane and the spirostene have a synergistic effect.

22. The composition of claim 20 wherein the spirostane is selected from the group consisting of laxogenin derivatives, 5-hydroxy-laxogenin derivatives and combinations thereof and the spirostene derivative comprises a 7-keto-diosgenin derivative.

23. The composition of claim 20 wherein the effective amount is preferably 0.01-5 mg./day and more preferably 0.1-1 mg./day.
